(19) United States
(12) Patent Application Publication
(10) Pub. No.: US 2008/0306572 A1
(43) Pub. Date: Dec. 11, 2008

(54) Apparatus for Experiencing Visual-Tactile Interaction

(76) Inventors: Yoshihisa Osada, Yamanashi (JP); Motoyasu Honma, Saitama (JP)

Correspondence Address: PAUL D. BIANCO;Fleit Gibbons Gutman Bongini & Bianco PL, 21355 EAST DIXIE HIGHWAY, SUITE 115, MIAMI, FL, 33180, US

(21) Appl. No.: 12/097,765

(22) PCT Filed: Nov. 29, 2007
(86) PCT No.: PCT/JP07/73053

§ 371 Date: Jun. 17, 2008

(30) Foreign Application Priority Data
Nov. 30, 2006 (JP) 2006-323044

(51) Int. Cl. A61N 1/00

(52) U.S. Cl. 607/54

(57) ABSTRACT

A subject is able to experience the sensation of a phantom limb by an effect of interaction generated between visual sensation and tactile sensation. A first chamber 110 for placing therein a prosthesis 305 that mimics a part of either a left or right limb, and a second chamber 160 arranged in parallel with the first chamber inside a case 105, are provided separated by a mirror 180 within the case. A stimulating unit 120, and a drive unit 125 for moving and/or rotating the stimulating unit, are provided within the first chamber. The subject is caused to experience the sensation of a phantom limb by inserting a limb on the side opposite the prosthesis into the second chamber, and viewing a mirror image 185 of the prosthesis placed in the first chamber through a viewing window 190.

### TECHNICAL FIELD

The present invention relates to an apparatus for experiencing visual-tactile interaction that allows a subject to experience the sensation of a phantom limb produced by interaction between tactile sensation and visual sensation.

### BACKGROUND ART

The phenomenon of phantom pain has been known in the past. A “phantom limb” refers to a phenomenon in which a person who has lost a portion of a limb, namely an arm (including the hand) or a leg (including the foot), receives a sensation as if the missing portion were actually present and the missing portion is functioning normally.

The concept of a phantom limb was experimentally confirmed by E. J. Milner and Gulland, et al. (see Non-Patent Document 1, for example).

Non-Patent Document 1 discloses an experiment conducted on subjects who had lost their left hand. In this experiment, the mirror image of the normal right hand of the subjects was reflected in a mirror, and the subjects were made to view the mirror image of their right hand.

According to this experiment, it was determined that the subjects felt as if their absent left hand was present and moving when they moved their right hand. Namely, it was determined that the subjects have experienced a phantom limb by perceiving the mirror image of their own right hand to be their missing left hand.

This experiment confirmed that the sensation of a phantom limb can also be produced through interactive effects between the senses (interactive effects between visual sensation and tactile sensation in the experiment of Non-Patent Document 1).

Non-Patent Document 1: Nature, Vol. 377, 12 Oct. 1995, pp. 489-490

### DISCLOSURE OF THE INVENTION

### Problems to be Solved by the Invention

However, since in the case of phantom pain, an affected area, namely the missing portion where the phantom pain is occurring, does not actually exist, it is impossible to massage and so on of the affected area. Consequently, there has been no effective treatment method for phantom pain.

An object of the present invention is to provide a solution to the above-mentioned problem. This object is achieved by combining characteristics described in the independent claims, while the dependent claims define more advantageous specific examples of the present invention.

Thus, according to the aspect of the present invention, there is provided an apparatus for allowing a subject to experience the sensation of a phantom limb (to be referred to as an apparatus for experiencing visual-tactile interaction) by generating interactive effects between the senses, and particularly interactive effects between visual sensation and tactile sensation.

### Means for Solving the Problems

An example of a configuration of the present invention is as described below. The apparatus for experiencing visual-tactile interaction in the invention described in claim 1 has a configuration comprising a case, a mirror, a stimulating unit, a drive unit and a viewing window.

The case has a first chamber provided inside the case, and a second chamber provided next to the first chamber inside the case.

The first chamber is a chamber for placing a prosthesis that mimics the part of either a left or right limb. A “limb” refers to an arm (including the hand) or leg (including the foot).

The second chamber is a chamber provided with an opening provided in the case for allowing a subject to insert a part of the other one of the limbs corresponding to that of the prosthesis. Furthermore, a “part corresponding to that of the prosthesis” refers to an actual part of the subject in the case of a normal subject, or to a missing part likened as if actually present in the case of a physically handicapped subject. Furthermore, a “normal subject” refers to a person in which all limbs are present without being missing. In addition, a “physically handicapped subject” (or simply a “handicapped subject”) refers to a person in which a portion of a limb is missing.

The stimulating unit is provided in the first chamber for giving (or imparting) a dynamic stimulus to the prosthesis.

In addition, the drive unit is provided in the first chamber for supporting the stimulating unit while also carrying out movement and/or rotation of the stimulating unit.

Furthermore, operation of this drive unit is electrically controlled by a control unit arranged inside or outside the first chamber.

The mirror is provided perpendicular to the bottom of the case between the first and second chambers for reflecting a mirror image of the prosthesis placed in the first chamber. Furthermore, the mirror image of the prosthesis is an image for causing the prosthesis to appear to a subject as a part of the other one of the limbs corresponding to that of the prosthesis.

In addition, the viewing window is provided in the case for allowing a subject to view the mirror image of the prosthesis reflected in the mirror. This viewing window is formed by cutting out in the approximately center of a front wall, back wall or top wall of the case.

According to this configuration, as a result of allowing a subject to view a mirror image of a prosthesis given a dynamic stimulus by the stimulating unit through the viewing window, the subject is allowed to experience the sensation of a phantom limb produced by interaction between visual sensation and tactile sensation. Furthermore, if the subject is to be allowed to experience the sensation of a phantom arm, this apparatus is placed on a table or desk, for example, so that the second chamber is roughly level. In this case, the subject uses the apparatus while sitting in a chair. In addition, if the subject is to be allowed to experience the sensation of a phantom leg, the apparatus is placed on a bed or mat, for example, so that the second chamber is roughly level. In this case, the subject uses the apparatus while getting on the bed or mat and sitting on the bed or mat.

According to the apparatus for experiencing visual-tactile interaction described in claims 2 to 6, the stimulating unit is preferably in the form of a contacting member that stimulates the prosthesis by direct contact in the manner of, for example, a brush, a fluid sprayer that stimulates by spraying a fluid in the form of, for example, wind such as air or liquid such as water, or a light projecting unit that stimulates by projecting light. According to this configuration, a subject can be shown a mirror image of a prosthesis to which is given a dynamic stimulus.

According to the apparatus for experiencing visual-tactile interaction described in claim 7, the first chamber is preferably provided with a blocking plate that conceals the portion of the drive unit so the portion of the drive unit is not reflected in the area of the mirror viewed from the viewing window as previously described. According to this configuration, the mechanism around the prosthesis, such as the drive unit, can be concealed from the field of view of a subject.

According to the apparatus for experiencing visual-tactile interaction described in claim 8, the drive unit is preferably provided with a support unit for supporting the stimulating unit, a rotation mechanism for turning (or rotating) the support unit (including a vibrating operation), a first plate-like member on which the rotation mechanism is installed for rotatably supporting the support unit, a second plate-like member coupled to one end of this first plate-like member, and a slide mechanism attached to this second plate-like member for moving the first plate-like member within a plane in parallel with the bottom of the case by means of the second plate-like member. According to this configuration, as a result of the drive unit causing the stimulating unit to carry out movement such as continuous movement, intermittent movement or reciprocating movement, or causing the stimulating unit to carry out turn such as revolution, rotation or oscillation within the range of a predetermined angle, a stimulus can be given by bringing the stimulating unit to a preferable location on the prosthesis.

According to the apparatus for experiencing visual-tactile interaction described in claim 9, the first plate-like member of the drive unit is preferably provided with a hole or groove for weight reduction. According to this configuration, the first plate-like member of the drive unit can be reduced in weight.

According to the apparatus for experiencing visual-tactile interaction described in claim 10, the opening of the second chamber is preferably provided at a location that allows a part of the other one of the limbs of the subject corresponding to that of a prosthesis to appear to be overlapping the mirror image of the prosthesis placed in the first chamber when the other one of the limbs is inserted into the second chamber through this opening. According to this configuration, the prosthesis placed in the first chamber can be made to appear to the subject as overlapping the part of the other one of the limbs corresponding to that of the prosthesis without causing a sense of incongruity.

According to the apparatus for experiencing visual-tactile interaction described in claim 11, the opening of the second chamber is preferably provided on the front side and the back side of the second chamber, respectively. According to this configuration, a subject is able to insert a limb inside the second chamber from either opening on the front side or the back side.

According to the apparatus for experiencing visual-tactile interaction described in claim 12, the prosthesis is preferably molded from a resin.

Moreover, according to the apparatus for experiencing visual-tactile interaction described in claim 13, the resin is a resin such as synthetic rubber or natural rubber having flexibility roughly the same as that of the skin of a limb of the body. According to this configuration, in the case the stimulating unit is formed in the form of a contacting member that stimulates a prosthesis by direct contact, or in the case the stimulating unit is formed in the form of a fluid sprayer that stimulates by spraying a fluid onto a prosthesis, the prosthesis is able to be smoothly deformed as if it were an actual part of the body as a result of the stimulating unit stimulating to the prosthesis.

According to the apparatus for experiencing visual-tactile interaction described in claim 14 or 15, a structure is employed in which the second chamber is symmetrical to the first chamber centering on the mirror.

Namely, according to the apparatus for experiencing visual-tactile interaction described in claim 14 or 15, there are preferably provided a case containing therein a first chamber and a second chamber arranged in parallel, a first stimulating unit within the first chamber for giving a dynamic stimulus to a first prosthesis placed in the first chamber that mimics a part of either a left or right limb, a first drive unit within the first chamber for supporting the first stimulating unit while also causing the first stimulating unit to move and/or turn, a second stimulating unit within the second chamber for giving a dynamic stimulus to a second prosthesis placed in the second chamber that mimics a part of the other left or right limb, a second drive unit provided within the second chamber for supporting the second stimulating unit while also causing the second stimulating unit to move, rotate or both, a first opening provided in the case for allowing a subject to insert a part of one of the limbs corresponding to that of the second prosthesis into the first chamber, a second opening provided in the case for allowing a subject to insert a part of the other one of the limbs corresponding to that of the first prosthesis into the second chamber, a mirror between the first chamber and the second chamber perpendicular to the bottom of the case, a first viewing window of the first chamber for allowing a subject to view a mirror image of the first prosthesis reflected in the mirror, and a second viewing window of the second chamber for allowing a subject to view a mirror image of the second prosthesis reflected in the mirror.

In the apparatus described in claim 14, the mirror is composed in the form of a mirror for reflecting both a mirror image of the first prosthesis and a mirror image of the second prosthesis, and (a) by allowing a subject who inserts a part of the other one of the limbs corresponding to that of the first prosthesis into the second chamber to view a mirror image of the first prosthesis given a dynamic stimulus by the first stimulating unit through the first viewing window, the subject is caused to experience the sensation of a phantom limb produced by visual-tactile interaction, or (b) by allowing a subject who inserts a part of one of the limbs corresponding to that of the second prosthesis into the first chamber to view a mirror image of the second prosthesis given a dynamic stimulus by the second stimulating unit through the second viewing window, the subject is caused to experience the sensation of a phantom limb produced by visual-tactile interaction.

In addition, in the apparatus described in claim 15, the mirror is composed in the form of a mirror for reflecting either a mirror image of the first prosthesis or a mirror image of the second prosthesis, and (a) when the prosthesis placed therein is the first prosthesis, by allowing a subject, who inserts a part of the other one of the limbs corresponding to that of the first prosthesis into the second chamber, with the mirror being attached to the case so as to oppose the first chamber, to view a mirror image of the first prosthesis given a dynamic stimulus by the first stimulating unit through the first viewing window, the subject is caused to experience the sensation of a phantom limb produced by visual-tactile interaction, or (b) when the prosthesis placed therein is the second prosthesis and the mirror is attached to the case so as to oppose the second chamber, by allowing a subject who inserts a part of one of the limbs corresponding to that of the second prosthesis into the first chamber, to view a mirror image of the second prosthesis given a dynamic stimulus by the second stimulating unit through the second viewing window, the subject is caused to experience the sensation of a phantom limb produced by visual-tactile interaction.

Thus, according to these configurations, a subject can be made to experience the sensation of a phantom limb by allowing the subject to insert a part of either the left or right limb into the apparatus from the same direction.

According to the apparatus for experiencing visual-tactile interaction described in claim 16, a cover is more preferably provided for selectively closing the first viewing window or the second viewing window. According to this configuration, the window not used to experience the sensation of a phantom limb can be selectively closed.

According to the apparatus for experiencing visual-tactile interaction described in claim 17, a cover is more preferably provided for selectively closing the first opening or the second opening. According to this configuration, the opening not used to experience the sensation of a phantom limb can be selectively closed.

### EFFECT OF THE INVENTION

According to the apparatus for experiencing visual-tactile interaction in the invention described in claim 1, the effects of visual-tactile interaction in the form of a prosthesis given a dynamic stimulus at a part of a missing limb of a subject can be experienced by the subject by allowing the subject to view a mirror image of a prosthesis given a dynamic stimulus by a stimulating unit through a viewing window. Particularly in the case a handicapped person is the subject, the sensation of a phantom limb can be experienced by visual-tactile interaction.

For example, this apparatus allows a person feeling phantom pain to experience a phantom limb at the missing portion where the phantom pain is occurring by the use thereof, and makes it possible for this person to undergo treatment for alleviating the phantom pain by giving a sensation in the manner of receiving a massage for the phantom limb. Accordingly, this apparatus can be used as a therapeutic apparatus for phantom pain.

In addition, this apparatus can be used not only as therapeutic apparatus for phantom pain, but also as an educational apparatus for allowing subjects such as normal persons or handicapped persons not suffering from phantom pain to experience the sensation of a phantom limb.

The apparatuses for experiencing visual-tactile interaction described in claims 2 to 17 additionally have the effects described below in addition to the effects of the apparatus described in claim 1.

According to the apparatuses for experiencing visual-tactile interaction described in claims 2 to 6, a subject can be shown a mirror image of a prosthesis given a dynamic stimulus. This mirror image is an image of the subject being passively stimulated. Consequently, the subject obtains a sensation as if receiving a massage from someone. Moreover, the subject receives the massage without being aware of the person performing the massage. Consequently, according to these configurations, the subject is able to receive the massage in a relaxed state, thereby making it possible to enhance effects in the form of rehabilitation.

According to the apparatus for experiencing visual-tactile interaction described in claim 7, the mechanism surrounding a prosthesis such as a drive unit and the like can be concealed from the field of view of the subject with a blocking plate. Thus, this apparatus is able to show the subject only a mirror image limited to the area closely surrounding the prosthesis when the subject looks into the first chamber through the viewing window. As a result, this apparatus enables subjects to focus their awareness on the mirror image of the prosthesis. Consequently, this apparatus enables a subject to efficiently experience a phantom limb.

According to the apparatus for experiencing visual-tactile interaction described in claim 8, a drive unit is able to carry out continuous movement, intermittent movement or reciprocating movement and the like of a stimulating unit, or carry out turn such as revolution, rotation or oscillation within the range of a predetermined angle. Thus, as a result of the drive unit carrying out a prescribed combination of these movement and turning (or rotating) operations, the stimulating unit can be brought to a preferable location on a prosthesis and a stimulus can be given at that location. As a result, this apparatus can produce a real tactile sensation, or in other words, a sensation as if actually being touched.

According to the apparatus for experiencing visual-tactile interaction described in claim 9, a first plate-like member of the drive unit can be reduced in weight. Consequently, this apparatus enables the first plate-like member to rapidly and smoothly carry out moving operation and/or turning operation (including a vibrating operation), while also enabling the drive source of the drive unit to reduced in size.

According to the apparatus for experiencing visual-tactile interaction described in claim 10, the prosthesis placed in the first chamber can be made to appear to the subject as overlapping the part corresponding to that of the prosthesis of the other limb without causing a sense of incongruity. Consequently, this apparatus enables a mirror image of a prosthesis to be perceived as an image of a part corresponding to that of a prosthesis of the subject's own other limb, thereby making it possible to facilitate experiencing the sensation of a phantom limb.

According to the apparatus for experiencing visual-tactile interaction described in claim 11, a subject is able to insert a limb into the second chamber from an opening in the front or and opening in the back. Consequently, this apparatus allows the sensation of both left and right phantom limbs to be experienced.

According to the apparatuses for experiencing visual-tactile interaction described in claims 12 and 13, a prosthesis is allowed to smoothly deform as if a part of the body as a result of the prosthesis being stimulated by the stimulating unit. Consequently, since this apparatus allows the subject to easily produce a tactile sensation, the subject is able to efficiently experience the sensation of a phantom limb.

According to the apparatuses for experiencing visual-tactile interaction described in claims 14 and 15, a subject is able to experience the sensation of a phantom limb by allowing the subject to insert a part of either a left or right limb into the apparatus from the same direction. Consequently, this apparatus enables a subject to experience the sensation of a phantom limb for a limb on either the left or right side with the subject positioned on the front side of the apparatus.

According to the apparatus for experiencing visual-tactile interaction described in claim 16, the viewing window not used to experience the sensation of a phantom limb can be blocked. Since a subject ends up being aware of a mirror image not used to experience the sensation of a phantom limb in the case of having seen that mirror image, the subject may not be able to fully experience the sensation of a phantom limb. Thus, the mirror image not used to experience the sensation of a phantom limb becomes an impairment to experiencing the sensation of a phantom limb. Consequently, this apparatus is able to conceal the mechanism not used to experience the sensation of a phantom limb from a subject by selectively blocking the viewing window not used to experience the sensation of a phantom limb. As a result, this apparatus allows a subject to preferably experience the sensation of a phantom limb at all times.

According to the apparatus for experiencing visual-tactile interaction described in claim 17, an opening not used to experience the sensation of a phantom limb can be selectively blocked. Consequently, this apparatus allows a subject to insert any part of a limb used to experience the sensation of a phantom limb into the proper opening at all times.

### BRIEF DESCRIPTION OF THE DRAWINGS

FIG. 1A and FIG. 1B are schematic drawings for respectively explaining the essential portions of an example of the configuration of an apparatus for experiencing visual-tactile interaction in a first embodiment of the present invention;

FIG. 2 is a drawing for explaining the essential portions of an example of the configuration of an apparatus for experiencing visual-tactile interaction in a first embodiment shown in FIG. 1;

FIG. 3 is a drawing for explaining the essential portions of an example of the configuration of an apparatus for experiencing visual-tactile interaction in a first embodiment shown in FIG. 1;

FIG. 4 is a drawing showing the configuration of an apparatus for experiencing visual-tactile interaction in a first embodiment shown in FIG. 1;

FIG. 5A and FIG. 5B are drawings for respectively explaining the essential portions of an example of the configuration of an apparatus for experiencing visual-tactile interaction in a first embodiment shown in FIG. 1;

FIG. 6 is a drawing for explaining the essential portions of an example of the configuration of an apparatus for experiencing visual-tactile interaction in a first embodiment shown in FIG. 1;

FIG. 7A to FIG. 7C are drawings for respectively explaining the essential portions of an example of the configuration of an apparatus for experiencing visual-tactile interaction in a second embodiment of the present invention;

FIG. 8 is a drawing for explaining the essential portions of an example of the configuration of an apparatus for experiencing visual-tactile interaction in a second embodiment shown in FIG. 7; and

FIG. 9 is a drawing for explaining the essential portions of an example of the configuration of an apparatus for experiencing visual-tactile interaction in a second embodiment shown in FIG. 7.

### BRIEF DESCRIPTION OF THE REFERENCE SYMBOLS

100: Apparatus for experiencing visual-tactile interaction
105: Case
110: First chamber
115: First flat surface
120: Stimulating unit
125: Drive unit
130: Control unit
160: Second chamber
165: Second flat surface
170: Opening
180: Mirror
185: Mirror image
190: Viewing window
305: Prosthesis
Da: Front
Db: Back
Dc: Top

### BEST MODE FOR CARRYING OUT THE INVENTION

The following provides an explanation of embodiments of the apparatus for experiencing visual-tactile interaction of the present invention with reference to the drawings. In each of the drawings, the same reference symbols are used to indicate those constituent features that are the same as common constituent features, and duplicate explanations thereof are omitted.

In addition, each drawing merely schematically indicates the shape, size and positional relationship of each constituent feature, and the present invention is not limited in any way to the examples shown in the drawings.

### Summary of the Invention

The apparatus for experiencing visual-tactile interaction as claimed in the present invention allows a subject to experience the sensation of a phantom limb produced by visual-tactile interaction by having the subject view a mirror image of a prosthesis which is being subjected to a dynamic stimulus through a viewing window. In this case, the subject may be a normal person or a handicapped person.

The apparatus for experiencing visual-tactile interaction in the present invention has a first chamber and second chamber inside a case. The first chamber is a chamber for placing a substitute for any part of a normal limb (to be simply referred to as a “limb”) which is an artificial limb (to be simply referred to as a “prosthesis”). Furthermore, a “limb” refers to an arm (including the hand) or a leg (including the foot). In addition, the second chamber is a chamber for inserting a limb on the opposite left or right side from the prosthesis. The first chamber is provided with a flat surface for placing the prosthesis. A mirror is vertically arranged between the first chamber and the second chamber on the bottom of the case. The mirrored side of the mirror faces the first chamber, while the back side, namely the opposite side from the mirrored side, faces the second chamber.

This apparatus has a stimulating unit that gives a dynamic stimulus to the prosthesis in the first chamber. In addition, this apparatus has a drive unit that supports the stimulating unit and causes the stimulating unit to move and/or turn (or rotate). Furthermore, the drive unit is provided with one or a plurality of three-dimensional movement mechanisms that allow the stimulating unit to freely move in the vertical and/or horizontal direction, and a rotation mechanism that allows the stimulating unit to make turning operations (including a vibrating operation). In addition, this apparatus has a viewing window in the portion of the case between the first chamber and the mirror.

This apparatus allows a subject to insert a limb for which the sensation of a phantom limb is to be experienced to be inserted into the second chamber and then view a mirror image of a prosthesis reflected in the mirror through the viewing window. Furthermore, in the case the subject is a normal person, the actual limb of that person is inserted into the second chamber.

In addition, in the case the subject is a handicapped person, the subject actually inserts the remaining portion of a missing limb through the opening into the second chamber if the limb is only missing to the elbow or knee. However, if the missing portion of the limb extends above the elbow or knee, the subject is unable to actually insert the missing limb through the opening into the second chamber. In this case, the subject assumes the missing limb to be present, namely forms an image thereof, and inserts that assumed missing limb through the opening into the second chamber.

This apparatus allows a subject to view a mirror image of a prosthesis through a viewing window. According to this apparatus, the apparatus switches to a mode for stimulating the prosthesis by driving the stimulating unit, and then shows a mirror image of the state of the prosthesis being stimulated by the stimulating unit to a subject. As a result, the apparatus enables a subject to experience the sensation of a phantom limb produced through interactive effects between the senses (here, between visual sensation and tactile sensation). Namely, this apparatus allows a subject to experience the sensation of a limb being stimulated even though it is not actually being stimulated.

### FIRST EMBODIMENT

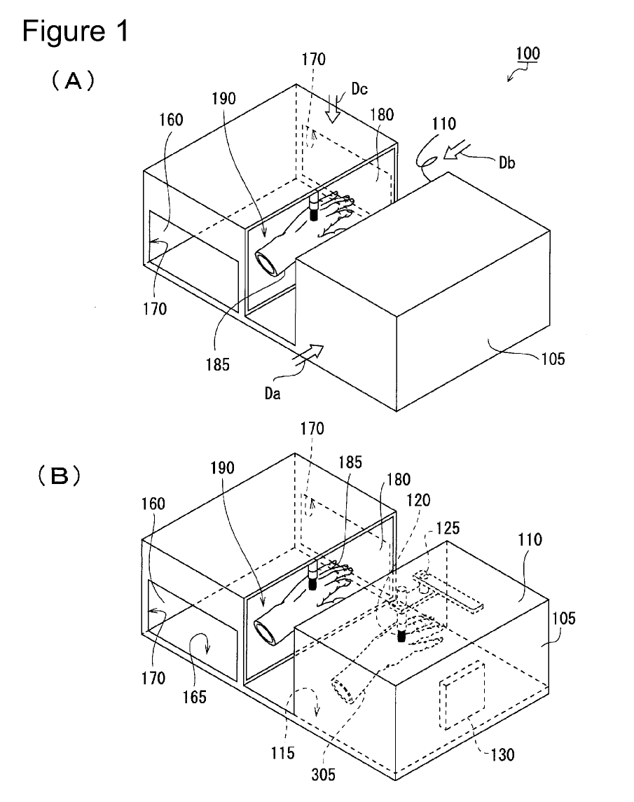

The following provides an explanation of the configuration of an apparatus for experiencing visual-tactile interaction in a first embodiment of the present invention with reference to FIGS. 1A and 1B and FIGS. 2 through 6. Furthermore, FIGS. 1A and 1B and FIGS. 2 through 6 are each drawings for explaining the configuration of an apparatus for experiencing visual-tactile interaction in a first embodiment. In the example of the configuration of this first embodiment, an explanation is provided of the case that the movement of the stimulating unit by the drive unit is linear.

FIG. 1A is an external view of an apparatus for experiencing visual-tactile interaction, while FIG. 1B indicates a portion of a schematic configuration of the inside of the apparatus for experiencing visual-tactile interaction with broken lines.

As shown in FIGS. 1A and 1B, the apparatus for experiencing visual-tactile interaction 100 (to be simply referred to as the “apparatus”) as claimed in this first embodiment is composed of a case 105 and various constituent features provided in the case 105. The case 105 has inside thereof a first chamber 110 and a second chamber 160 divided by walls and partitions of the case 105. The first chamber 110 and the second chamber 160 are arranged in parallel. Although there are no particular limitations on the shape of the case 105, it preferably has the shape of a hexahedron. In addition, the case 105 preferably uses a mirror 180 to be described later as a partition.

The following provides an explanation using the example of a subject experiencing the sensation of a phantom arm.

The first chamber 110 is a chamber for placing a prosthesis 305 that mimics either a left or right limb. A prosthesis of the right hand is shown in the example shown in FIGS. 1A and 1B.

The first chamber 110 is provided with a flat bottom (to be referred to as a “first flat surface”) 115 of the case 105 on which the prosthesis 305 is placed, a stimulating unit 120 for giving a dynamic stimulus to the prosthesis 305 placed on the first flat surface 115, and a drive unit 125 that supports the stimulating unit 120 and allows the stimulating unit 120 to make moving operation and/or turning operation (including a vibrating operation). The operation of the drive unit 125 is electrically controlled by a control unit 130. The control unit 130 may be arranged at a location either inside or outside the first chamber 110.

The second chamber 160 is a chamber for allowing a subject to insert the other limb, namely a limb on the opposite side from the prosthesis 305. Furthermore, since the prosthesis 305 is the right hand in the example shown in FIGS. 1A and 1B, the limb on the opposite side is either the left hand or left arm. The second chamber 160 is provided with a flat bottom (to be referred to as a “second flat surface”) 165 of the case 105 on which the subject's limb is placed. In this configuration example, the second flat surface 165 and the first flat surface 115 lie within the same plane.

As shown in FIGS. 1A and 1B, the second chamber 160 is provided with an opening 170, and this opening 170 is provided in the case 105 for inserting the limb on the opposite side from the prosthesis 305.

This opening 170 is respectively provided on the front Da and back Db of the case 105. As a result, a subject can insert a limb into the second chamber 160 from either an opening 170 in the front Da or an opening 170 in the back Db.

As previously described, a mirror 180, and preferably a flat mirror, is provided between the first chamber 110 and the second chamber 160 to reflect a mirror image 185 of the prosthesis 305 placed in the first chamber 110.

In addition, a viewing window 190 for allowing a subject to view the mirror image 185 of the prosthesis 305 reflected in the mirror 180 is provided in the case 105.

In this configuration example, although the viewing window 190 is formed by respectively cutting out a portion roughly in the center of a wall on the front Da, a wall on the back Db and a wall on the top Dc of the case 105, the viewing window is not limited thereto. This viewing window 190 enables a subject to view roughly all or a portion of the surface of the mirror 180 facing the first chamber 110. In addition, this viewing window 190 is preferably provided so that a portion of the boundary of the viewing window 190 is within the same plane as the mirrored surface of the mirror 180 or adjacent thereto. This mirror 180 is preferably provided perpendicular to the first flat surface 115 and the second flat surface 165.

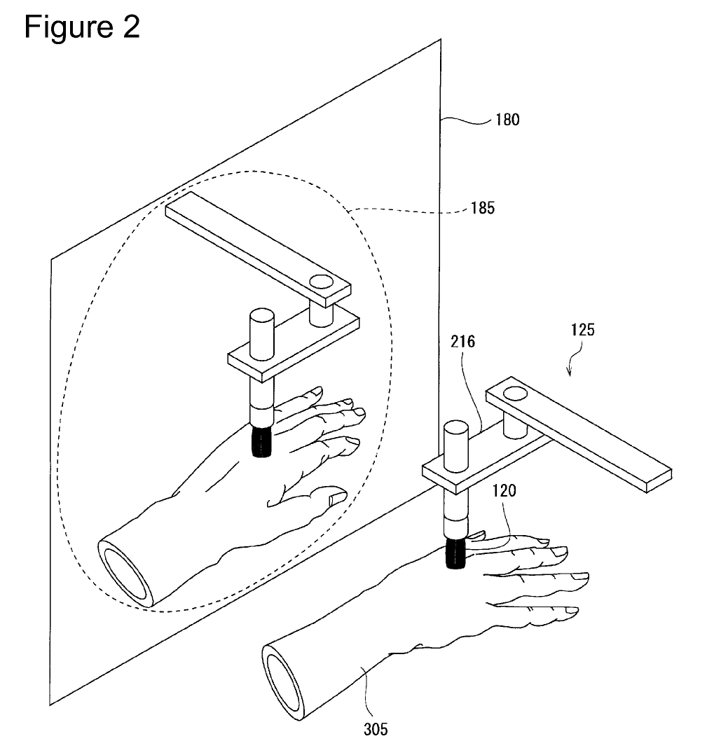

FIG. 2 is a drawing indicating a simplified representation of the configuration of the drive unit and showing the relationship between prosthesis 305 and a mirror image 185 of the prosthesis 305 reflected in mirror 180. FIG. 2 shows the state in which the stimulating unit 120 is stimulating the prosthesis 305. This stimulating unit 120 is preferably composed in the form of a means for stimulating the prosthesis 305 with, for example, a rod-like object, brush-like object, light or fluid.

In the case of using a rod-like object as the stimulating unit 120, the distal end that stimulates is preferably in the form of a flat surface, curved surface or needle, and can be of any shape corresponding to the design. In the case of stimulating the prosthesis 305 with a rod-like object, chiropractic effects or acupuncture effects can be expected to be given by visual-tactile interaction to a subject viewing a mirror image 185 of the state of the rod-like object applying pressure to the prosthesis 305 by making direct contact therewith.

In addition, in the case of stimulating the prosthesis 305 with a brush-like object, gentle massaging effects can be expected to be similarly given by visual-tactile interaction to the subject.

In addition, in the case of stimulating the prosthesis 305 with light, moxibustion effects or bathing effects can be expected to be given by visual-tactile interaction to a subject viewing a mirror image 185 of the state of light from the stimulating unit 120 stimulating the prosthesis 305 by shining, or being irradiated, thereon.

In addition, in the case of stimulating the prosthesis 305 with a fluid such as wind or a liquid, chiropractic effects, moxibustion effects or bathing effects can be expected to respectively given by visual-tactile interaction to a subject. In this case, stimulating unit 120 preferably employs a configuration that stimulates the prosthesis 305 by adjusting the intensity of the wind or the flow rate of the liquid. Furthermore, the prosthesis 305 is preferably formed from a resin such as synthetic rubber or natural rubber having flexibility roughly the same as that of the skin of a limb of the body so as to be deformed in the case of being stimulated from the stimulating unit 120. As a result, this apparatus 100 enables the prosthesis 305 to be deformed as it were a part of the body as a result of the prosthesis 305 being stimulated by wind, for example.

In the example shown in FIG. 2, the stimulating unit 120 is composed using a writing brush, for example, for the brush. The stimulating unit 120, composed as a brush, stimulates the prosthesis 305 in direct contact therewith. Furthermore, as was previously described, the stimulating unit 120 may also be composed in the form of a fluid sprayer, which stimulates by spraying wind such as air or a liquid such as water, provided with an adjustment unit for flow rate and/or flow pressure, for example, instead of a brush. Furthermore, a flow rate or flow pressure adjustment unit can be composed by providing a valve, for example, in a flow path through which a fluid flows, as is commonly known. Alternatively, the stimulating unit 120 may be composed in the form of a light projecting unit, which stimulates by projecting light, provided with a light quantity adjustment unit. A light quantity adjustment unit guides light to the same light path by increasing a voltage applied to a light source or the number of emission sources, as is commonly known. Furthermore, in the case of composing the stimulating unit 120 in the form of a light projecting unit, a configuration is preferably employed that projects light in the manner of an LED light using an LED for the light source or an optic fiber that guides light from an emission source.

This stimulating unit 120 is supported by the drive unit 125. This drive unit 125 can be made to move and/or turn the stimulating unit 120. As a result, in the case of using a brush-like object for the stimulating end of the stimulating unit 120, for example, the brush-like object gives a dynamic stimulus to the prosthesis 305 in the form of rubbing the prosthesis 305. Furthermore, although the movement of this stimulating unit 120 consists of a combination of linear movement and turn movement in this first embodiment, this movement is not limited thereto, but rather the stimulating unit 120 is also able to move in any desired manner as dictated by the design among one-dimensional, two-dimensional and three-dimensional movement in addition to turn movement. In addition, turn can include revolution and/or rotation corresponding to the design.

The drive unit 125 preferably rigidly supports the stimulating unit 120 with a support unit 216 and a first plate-like member 214a to be described later so that the stimulating unit 120 does not shake in an unstable state. The support unit 216 is preferably formed to be screwed in or clamped to removably and rigidly support the stimulating unit 120.

The mirror 180 reflects therein a mirror image 185 of the prosthesis 305 which is being subjected to a dynamic stimulus by the stimulating unit 120.

This apparatus 100 allows a subject to experience the sensation of a phantom limb produced by visual-tactile interaction by allowing the subject to view the mirror image 185 of the prosthesis 305 which is being subjected to a dynamic stimulus by the stimulating unit 120 through the viewing window 190 (see FIG. 1).

Furthermore, as was previously explained, the prosthesis 305 is preferably formed with a resin such as synthetic rubber or natural rubber having flexibility roughly the same as that of the skin of a limb of the body so as to be deformed in the case of being stimulated by the stimulating unit 120. As a result, this apparatus 100 allows the prosthesis 305 to be deformed as if it were a part of the body as a result of being stimulated by the stimulating unit 120 in the case of the stimulating unit 120 being formed in the form of a contacting member that stimulates by making direct contact with the prosthesis 305, or in the case of being formed in the form of a fluid sprayer that stimulates to the prosthesis 305 by spraying a fluid. Consequently, since this prosthesis 305 facilitates the generation of a stronger visual-tactile interaction for a subject, the subject is able to experience the sensation of a phantom limb efficiently.

Furthermore, in the example shown in FIGS. 1 and 2, the prosthesis 305 is composed in the form of a rubber hand. In addition, as was previously explained, this prosthesis 305 is on the opposite left or right side of the limb for which the subject is to experience the sensation of a phantom limb.

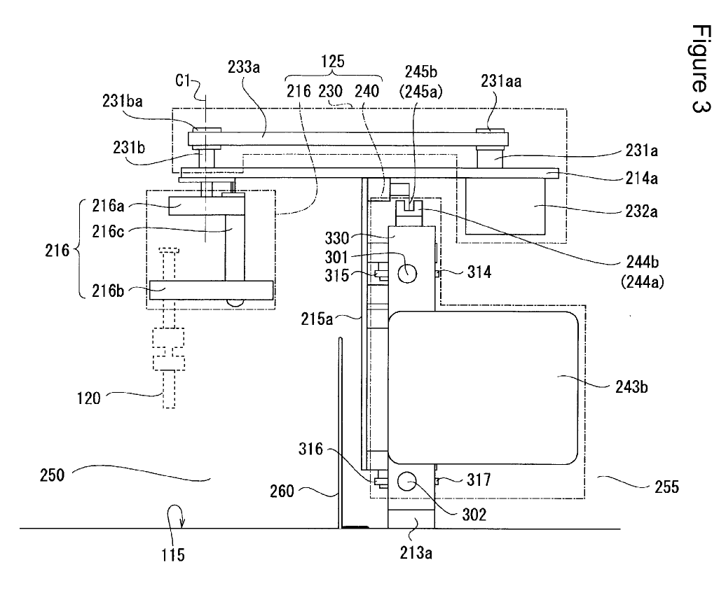

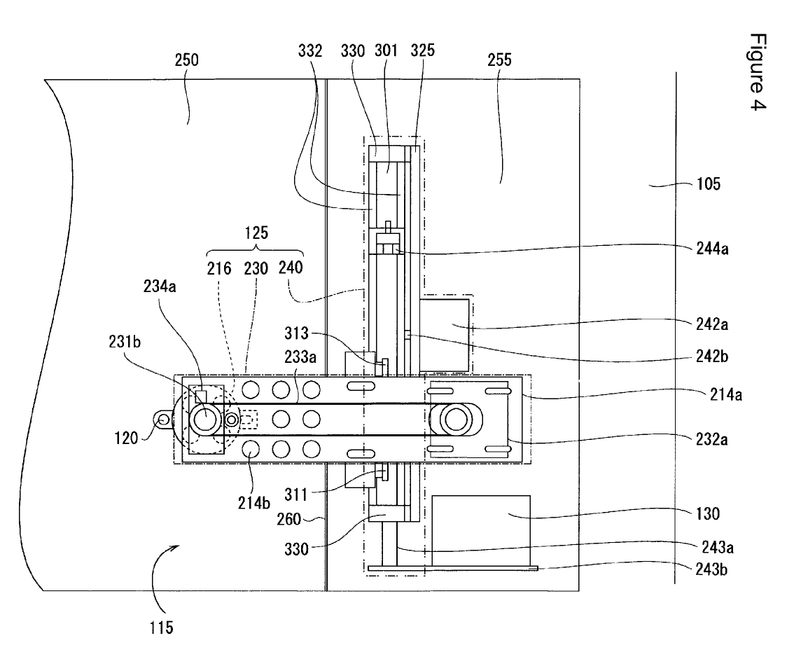

FIGS. 3 and 4 show a specific example of the configuration of the stimulating unit 120 and the drive unit 125. FIG. 3 shows the configuration of the stimulating unit 120 and the drive unit 125 as viewed from the front Da (see FIG. 1A), while FIG. 4 shows the configuration of the stimulating unit 120 and the drive unit 125 as viewed from the top Dc (see FIG. 1A).

Furthermore, in the example shown in FIGS. 3 and 4, the stimulating unit 120 is indicated with dotted lines. This indicates that the stimulating unit 120 can be removed from the first plate-like member 214a. This apparatus 100 secures a large space allowing the insertion of a limb of a subject by removing the stimulating unit 120 from the drive unit 125. As a result, this apparatus 100 enables a subject to easily insert a limb into the second chamber 160.

Furthermore, in the example shown in FIGS. 3 and 4, a single slide mechanism 240 for performing linear movement is shown as an example of a movement mechanism. In addition, a single rotation mechanism 230 is shown in the example shown in FIGS. 3 and 4.

As shown in FIGS. 3 and 4, the drive unit 125 is arranged on the first flat surface 115 by a foundation 213a that supports the entirety of the drive unit 125. Furthermore, the area on the first flat surface 115 of the first chamber 110 is divided by a blocking plate 260 into a prosthesis placing space 250 for placing the prosthesis 305, and a drive unit arranging space 255 for arranging the drive unit 125. This blocking plate 260 is vertically fixed on the first flat surface 115. This blocking plate 260 is a plate for concealing the drive unit 125 so that the drive unit 125 is not reflected in the mirror. This blocking plate 260 conceals the drive unit 125 and other mechanisms surrounding the prosthesis 305 from the field of view of a subject, while also blocking the portion of those mechanisms so that they do not appear in the mirror 180. Thus, this apparatus 100 allows a subject to view only a mirror image 185 that is limited to only the extreme vicinity of the prosthesis 305 when viewing the inside of the first chamber 110 through the viewing window 190. As a result, this apparatus 100 allows subjects to focus their awareness on the mirror image 185 of the prosthesis 305. Consequently, this apparatus 100 allows a subject to efficiently experience the sensation of a phantom limb.

The drive unit 125 mainly comprises a support 216 for supporting the previously described stimulating unit 120, a rotation mechanism 230, a slide mechanism 240, and a first plate-like member 214a and a second plate-like member 215a formed in the shape of long strips mutually joined in the shape of the letter T. These first and second plate-like members 214a and 215a are preferably in the form of plate-like objects.

In the configuration example shown in FIG. 3, the support unit 216 has first and second plate-like objects 216a and 216b mutually separated in parallel while axially aligned with a central axis C1, and a coupling rod 216c. The coupling rod 216c is provided in parallel with the central axis C1, and mutually couples the first and second plate-like objects 216a and 216b at positions offset from the central axis C1. The above-mentioned stimulating unit 120 is attached at a position removed from the central axis C1 at a different location from the coupling rod 216c of the second plate-like object 216b. On the other hand, the first plate-like object 216a is rigidly connected to a second rotating shaft 231b of the rotation mechanism 230 on the central axis C1. Thus, the first and second plate-like objects 216a and 216b turn, or in other words, rotate about the central axis C1 in response to rotation of the second rotating shaft 231b. As a result, the stimulating unit 120 attached to the second plate-like object 216b revolves around the central axis C1.

The rotation mechanism 230 is substantially mounted on the above-mentioned first plate-like member 214a. This first plate-like member 214a has mutually parallel top and bottom surfaces, and is provided in parallel with the first flat surface, namely the bottom, 115 of the case 105. The second rotating shaft 231b described above is rotatably held on one end of the first plate-like member 214a by means of a bearing not shown. A second belt pulley 231ba having surface irregularities in the manner of a gear is provided on the second rotating shaft 231b on the opposite end of the support unit 216. On the other hand, a rotary drive motor 232a is attached to the other end of the first plate-like member 214a beneath the first plate-like member 214a. The rotating shaft of this motor 232a, namely a motor shaft (not shown), is removably and rigidly connected to the first rotating shaft 231a attached to the upper surface of the first plate-like member 214a. In this case, the motor shaft or the first rotating shaft 231a is rotatably held on the first plate-like member 214a by means of a bearing not shown. Moreover, a first belt pulley 231aa having surface irregularities in the manner of a gear is provided on this first rotating shaft 231a on the opposite side from the side connected to the motor shaft in the same manner as the case of the second rotating shaft 231b.

Moreover, an endless belt 233a is provided between the first and second belt pulleys 231aa and 231ba. Surface irregularities that engage with the belt pulleys are provided in this endless belt 233a.

As a result of being composed in the manner described above, the rotation mechanism 230 transfers rotational movement to the first rotating shaft 231a, the endless belt 233a and the second rotating shaft 231b accompanying rotation of the rotary drive motor 232a. Accordingly, the support 216 rotates and as a result thereof, the stimulating unit 120 revolves. In addition, the rotation mechanism 230 has a rotation positioning sensor 234a.

Next, an explanation is provided of a slide mechanism, or in other words, a movement mechanism, of the drive unit 125.

This slide mechanism 240 is connected to the second plate-like member 215a, and this second plate-like member 215a is connected perpendicular to the lower surface of the first plate-like member 214a. The location where this second plate-like member 215a is attached to the first plate-like member 214a is preferably a location that allows the obtaining of a mechanical equilibrium so that the first plate-like member 214a does not deviate from a location parallel to the bottom 115.

Prior to explaining the connecting relationship between the second plate-like member 215a and the slide mechanism 240, an explanation is first provided of the main components of the slide mechanism with reference to FIGS. 3, 4, 5A, 5B and 6.

The slide mechanism 240 mainly has mutually parallel first and second guide rods 301 and 302, first, second, third, fourth, fifth, sixth and seventh slide rollers 311, 312, 313, 314, 315, 316 and 317 that rotate by contacting these guide rods, a slide drive motor 242a, a large-diameter drum 320, first and second guide rollers 321 and 322, and, wires of suitable strength that are not broken easily, such as first and second piano wires 323 and 324, one end each thereof is fastened to the large-diameter drum 320 while the other end is fastened to the second plate-like member 215a.

The respective cross-sectional shapes of the above-mentioned first and second guide rods 301 and 302 are preferably circular or quadrilateral, although not limited thereto. However, the surfaces thereof are preferably smooth surfaces. These first and second guide rods 301 and 302 are fastened to a pair of guide rod supports 330,330 fastened perpendicular to the bottom 115 of the case 105. Both ends of each of these first and second guide rods 301 and 302 are fastened to the pair of guide rod supports 330, 330 and arranged in parallel with the bottom 115 of the case 105 and the second plate-like member 215a. In addition, a support plate 243b is attached to at least one of the guide rod supports 330, 330 by means of a support rod 243a.

The above-mentioned second plate-like member 215a is supported by the first guide rod 301 and the first through fifth slide rollers 311 to 315, while also being supported by the second guide rod 302 and the sixth and seventh slide rollers 316 and 317. Namely, the first through seventh slide rollers 311 to 317 are composed using, for example, roller bearings, and each stationary shaft thereof axially aligned with the central axis is connected to the second plate-like member 215a. Thus, roller bearings installed with these stationary shafts 311a, 312a, 313a, 314a, 315a, 316a and 317a, namely each of the slide rollers 311 to 317, enable movement of the second plate-like member 215a while supporting the second plate-like member 215a by rolling over the surfaces of the first and second guide rods 301 and 302. In this case, for example, the first and third slide rollers 311 and 313 contact the upper surface of the first guide rod 301. In addition, the second slide roller 312 contacts the lower surface of the first guide rod 301. Moreover, the fourth and fifth slide rollers 314 and 315 contact mutually opposite sides of the first guide rod 301. Similarly, the sixth and seventh slide rollers 316 and 317 contact mutually opposite sides of the second guide rod 302.

In this manner, in addition to the second plate-like member 215a being perpendicularly held on the bottom 115 of the case 105 by the seven slide rollers 311 to 317 rolling over these two guide rods 301 and 302, the second plate-like member 215a is able to move in a direction parallel to the bottom 115 within a plane perpendicular to the bottom 115.

Furthermore, although there are no particular limitations on the planar shape of the second plate-like member 215a, in this embodiment, the upper area on the side that joins with the first plate-like member 214a has a wide rectangular shape, and the area below the vicinity where the second slide roller 312 is connected has a narrow, long rectangular shape that is narrower than the upper area for the purpose of reducing the weight of the second plate-like member 215a.

In addition, in this embodiment, the stationary shafts 311a, 312a and 313a are attached to auxiliary members 311b, 312b and 313b fastened to amounting surface of the second plate-like member 215a and are attached perpendicular to the mounting surface of the second plate-like member 215a. On the other hand, the stationary shafts 314a, 315a, 316a and 317a are attached to auxiliary members 314b and 316b fastened while protruding perpendicularly from the mounting surface of the second plate-like member 215a at an orientation and are attached perpendicular to the bottom 115 of the case 105. Furthermore, in this configuration example, the stationary shaft 317a is attached to the auxiliary member 316b in common with the stationary shaft 31a.

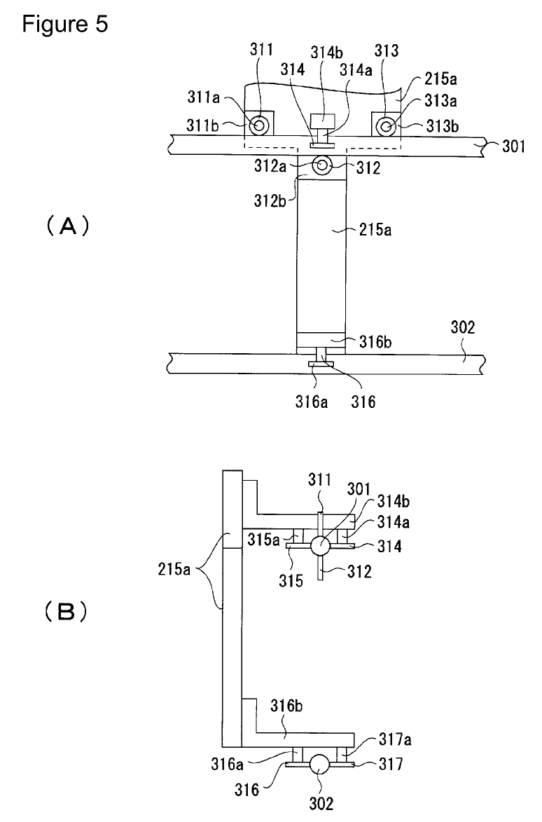

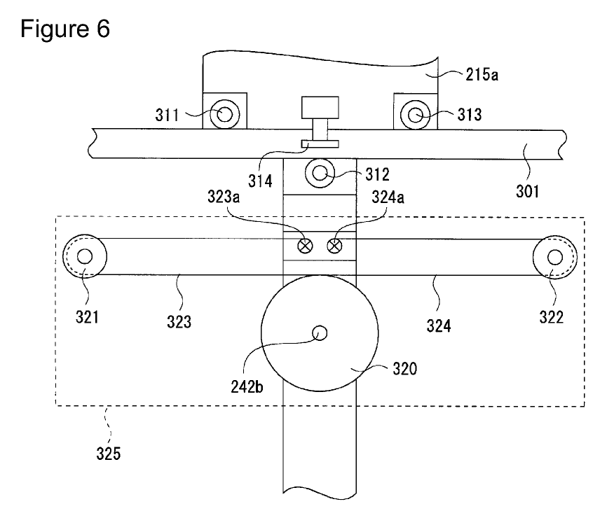

Next, an explanation is provided of a mechanism for transferring drive force of the slide mechanism 240 with reference to FIGS. 5A, 5B and 6.

In this embodiment, a plate-like auxiliary plate 325 (indicated with broken lines) is fastened between the above-mentioned guide rod supports 330, 330 perpendicular to the bottom 115 of the case 105, and in parallel with the second plate-like member 215a. This auxiliary plate 325 is preferably provided between the first and second guide rods 301 and 302. A rotating shaft (motor shaft) 242b of the slide drive motor 242a is rotatably fastened to this auxiliary plate 325 by means of a bearing. The large-diameter drum 320 is removably fastened to this motor shaft 242b. Thus, this drum 320 is arranged between the second plate-like member 215a and the auxiliary plate 325.

Moreover, first and second guide rollers 321 and 322 having a diameter smaller than that of the drum 320 are attached to this auxiliary plate 325 on the same side as the large-diameter drum 320. In this case, the central axes of the first and second guide rollers 321 and 322 are parallel to the motor shaft 242b and are respectively attached at symmetrical locations in terms of distance from the motor shaft 242b.

On the one hand, fastening units 323a and 324a for fastening the other ends of first and second piano wires 323 and 324 are provided on the previously described second plate-like member 215a, protruding from the mounting surface of the second plate-like member 215a towards the auxiliary plate 325 without colliding with the drum 320. On the other hand, each of the other ends of the first and second piano wires 323 and 324 is fastened on the outer periphery of the drum 320, and the first and second piano wires 323 and 324 are suspended between the drum 320 and the fastening units 323a and 324a by means of first and second guide rollers 321 and 322, respectively. As a result, when slide drive motor 242a is started and motor shaft 242b rotates, large-diameter drum 320 rotates, first and second piano wires 323 and 324 are pulled in one direction according to the direction of this rotation, and as a result, the second plate-like member 215a moves in that direction. Furthermore, portions of the first and second piano wires 323 and 324 are naturally wrapped around the periphery of the large-diameter drum 320 by an amount sufficient for allowing the second plate-like member 215a to be moved by the rotation of the large-diameter drum 320. Whereupon, the second plate-like member 215a is able to slide over a long distance with only a small rotation angle or amount of rotation by the large-diameter drum 320.

Furthermore, the important constituent of the slide mechanism 240 as described above is merely an example thereof, and it should be clearly understood that this is not limited in any way thereto.

Furthermore, in the configuration example shown in FIGS. 3 and 4, the first plate-like member 214a is a member that supports the rotation mechanism 230 in the horizontal direction. In addition, the second plate-like member 215a is a member that supports the rotation member 230 in the vertical direction.

The movement mechanism in the form of slide mechanism 240 is further provided with first and second sensors 244a and 244b for stopping movement or reversing movement by detecting the slide position of the second plate-like member 215a moved by the slide drive motor 242a. These first and second sensors 244a and 244b are provided on two parallel cleats provided between the apices of the guide rod supports 330, 330, namely on bridge 332. Although the interval between these first and second sensors 244a and 244b is equal to the distance over which the second plate-like member 215a moves, that interval can be arbitrarily set corresponding to the design. In addition, the central axis of the motor shaft 242b described above, or an line extending there from, is made to be positioned directly below the midpoint between the first and second sensors 244a and 244b. These first and second sensors 244a and 244b preferably comprise, for example, optical sensors combining a light emitting device and a light receiving device, and are composed so as to be turned on and off by first and second blocking plates 245a and 245b coupled to the second plate-like member 215a and provided corresponding to the first and second sensors 244a and 244b, respectively. This configuration combining these first and second sensors 244a and 244b with the first and second blocking plates 245a and 245b can be easily composed by a person with ordinary skill in the art.

In the configuration example of the first embodiment as described above, the rotary drive motor 232a, the slide drive motor 242a and the first and second sensors 244a and 244b are respectively connected to the control unit 130 that carries out electrical control.

This control unit 130 is connected to an ordinary 100 V power supply, and is provided with a main switch (not shown) for switching the power supply of the apparatus 100 in the present embodiment on and off. Moreover, this control unit 130 is respectively provided with an operating switch (not shown) for starting and stopping each motor 232a and 242a. Moreover, this control unit 130 is provided with a so-called mode selection switch capable individually selecting and setting rotating speed, flow rate or quantity of light and so forth corresponding to the constitution of the stimulating unit 120.

In addition, this control unit 130 is provided with a mode selection switch that switches the first and second switches 244a and 244b on, and makes these first and second switches 244a and 244b stop the slide drive motor 242a or reverse this motor 242a in response that the light from the light receiving element is blocked by the first and second blocking plates 245a and 245b and does not reach a light receiving element.

Since such a control unit 130 can be easily composed by a person with ordinary skill in the art using conventionally known technology, a detailed explanation thereof is omitted.

The drive unit 125 explained in the first embodiment described above is composed with the rotation mechanism 230 and the slide mechanism 240. The slide mechanism 240 is a mechanism for sliding the second plate-like member 215a, and ultimately the stimulating unit 120, linearly in one or both directions.

However, this drive unit 125 may be composed, if possible, by using a robot arm resembling an industrial robot arm used in various types of industry. In addition, although the slide mechanism 240 can be composed in the form of a mechanism capable of sliding in two or three dimensions, these mechanisms can be easily designed by a person with ordinary skill in the art. Furthermore, the drive unit 125 is preferably provided with measures such as reduced weight, reduced size, quiet operation and low energy consumption so as to prevent a subject from being aware of the presence thereof. An example, in the first embodiment, of such measures consists of the drive unit 125 being provided with a plurality of weight reduction holes 214b in the first plate-like member 214a. Furthermore, the drive unit 125 may also employ a configuration that provides grooves instead of the weight reduction holes 214b.

In addition, the rotation mechanism 230 is provided with a motor 232a for causing turning operation (including a vibrating operation) of the stimulating unit 120, and a rotary drive belt 233a for transferring driving of the rotary drive motor 232a to the stimulating unit 120 as previously described. In the case of this rotation mechanism 230, when a switch for operating the rotary drive motor 232a of the control unit 130 (see FIG. 1) is switched on, rotary drive motor 232a rotates, and this rotation is transferred to the first rotating shaft 231a, the first belt pulley 231aa, the endless belt 233a, the second belt pulley 231ba and the second rotating shaft 231b, thereby causing the support 216 to rotate about central axis C1, which in turn causes rotary movement to the stimulating unit 120 attached to this support 216.

On the other hand, in the case of the slide mechanism 240, when a slide control switch of the control unit 130 is switched on, the slide drive motor 242a rotates. This rotation is converted to linear motion of the first and second piano wires 323 and 324 through the rotary movement of the motor shaft 242b and the drum 320, and then transferred to the second plate-like member 215a. Accordingly, the second plate-like member 215a moves linearly guided by the first and second guide rods 301 and 302 by a distance corresponding to the amount of rotation of the motor 242a, and thus the amount of rotation of the large-diameter drum 320. Since this second plate-like member 215a is coupled to the first plate-like member 214a, this first plate-like member 214a and the support 216 coupled thereto, and thus the stimulating unit 120 attached thereto, naturally also carry out linear movement similar to that of the second plate-like member 215a. In this case, the stimulating unit 120 can be set at a required location by finely adjusting the linear movement in one or both directions of the second plate-like member 215a by operating the slide mode selector switch. In addition, the rotation of the stimulating unit 120 can be controlled to a required form of rotation by similarly operating the rotation mode selector switch of the control unit 130.

This apparatus 100 produces the effect of an interaction between senses (here, between visual sensation and tactile sensation). This interaction between senses is also produced even if a limb is missing. Consequently, in the case of assuming to be used by a handicapped person missing a portion of a limb, this apparatus 100 allows the subject to experience the sensation of that missing portion. At this time, this apparatus 100 activates a sensory region in the brain of the subject that governs the missing portion. Consequently, this apparatus 100 can allow, for example, the subject to experience the sensation of receiving treatment such as a massage at that missing portion as a result of massaging the prosthesis 305. As a result, in the case the subject is a person experiencing phantom pain, for example, this apparatus 100 is able to achieve rehabilitation effects, namely effects that relieve the phantom pain of the subject. Thus, this apparatus 100 is able to perform treatment in the form of relieving phantom pain of a subject.

If this apparatus 100 is to allow a subject to experience the sensation of a phantom arm, then the apparatus 100 is placed on a table or desk, for example, so that the second chamber 160 is roughly level. In this case, the subject uses the apparatus while sitting in a chair. In addition, if this apparatus is to allow a subject to experience the sensation of a phantom leg, then the apparatus 100 is placed on a bed or mat so that the second chamber 160 is roughly level. In this case, the subject gets onto the bed or mat and then uses the apparatus while sitting up on the bed or mat. Furthermore, the limb on the left side is inserted in the case of inserting a limb into opening 170 on the front Da, while the limb on the right side is inserted in the case of inserting a limb into the opening on the back Db. Thus, this apparatus 100 enables a subject to experience the sensation of a phantom limb on both the left and right sides of the body with a single apparatus.

This apparatus 100 has the following characteristics to facilitate experiencing the sensation of a phantom limb by a subject.

(1) In the experiment disclosed in the previously described Non-Patent Document 1, since the normal limb of a subject was used as a target for stimulating, within the brain of the subject, the stimulus only acted on the sensory region of the normal limb sometimes, and the stimulus did not act on the sensory region of a missing portion of a limb so as to experience the sensation of a phantom limb. Consequently, according to the experiment disclosed in Non-Patent Document 1, the subject was sometimes unable to experience the sensation of a phantom limb. As a result, in the experiment disclosed in Non-Patent Document 1 as described above in which the subject is sometimes unable to experience the sensation of a phantom limb.

In contrast, this apparatus 100 uses the prosthesis 305 as the target of stimulating instead a limb of the subject him or herself. Moreover, since this prosthesis 305 is formed from a resin such as synthetic rubber or natural rubber having flexibility similar to the skin of an actual limb, the stimulating causes a reaction that is similar to that of the skin of an actual limb. Consequently, this apparatus 100 easily allows a subject to experience the sensation of a phantom limb.

(2) This apparatus 100 has a stimulating unit 120 for giving a dynamic stimulus to the prosthesis 305, a drive unit 125 for supporting and operating the stimulating unit 120, and a control unit 130 for controlling operation of the drive unit 125. This apparatus 100 allows a subject to view a mirror image 185 of the prosthesis 305 through a viewing window 190. At this time, this apparatus 100 operates the stimulating unit 120 by driving the drive unit 125 with the control unit 130. As a result, this apparatus 100 allows the stimulating unit 120 to give to the prosthesis 305 a smooth dynamic stimulus as if being rubbed by an actual person. As a result, this apparatus 100 is able to easily produce an interaction between the visual sensation and tactile sensation of the subject, thereby enabling the subject to easily experience the sensation of a phantom limb.

(3) This apparatus 100 blocks the mechanism such as the drive unit 125 surrounding the prosthesis 305 from the view of the subject with the blocking plate 260. Thus, this apparatus 100 allows the subject to view only the mirror image 185 limited to only the extreme vicinity of the prosthesis 305 when the subject views the inside of the first chamber 110 through the viewing window 190. As a result, this apparatus 100 enables subjects to focus their awareness on the mirror image 185 of the prosthesis 305. Consequently, this apparatus 100 allows the subject to efficiently experience the sensation of a phantom limb.

(4) The experiment disclosed in Non-Patent Document 1 does not allow the experiment to be performed on persons missing limbs on both sides of the body. Consequently, the experiment disclosed in Non-Patent Document 1 does not allow such persons to experience the sensation of a phantom limb.

In contrast, this apparatus 100 is able to allow persons missing limbs on both sides of the body to experience the sensation of a phantom limb, and is therefore effective for such persons.

(5) This apparatus 100 has, for example, weight reduction holes 214b (which may also be in the form of grooves) in the first plate-like member 214a of the drive unit 125 for the purpose of realizing reduced weight, reduced size, quiet operation and reduced energy consumption and so on of the apparatus. Since the first plate-like member 214a of the drive unit 125 can be reduced in weight, the apparatus 100 is able to rapidly and smoothly carry out any one or more operations of moving, turning operations (including a vibrating operation) the first plate-like member 214a in one or both directions, while also making it possible to reduce the size of the drive unit 125. Consequently, this apparatus 100 makes it easy for a subject to concentrate on the prosthesis 305.

### SECOND EMBODIMENT

This second embodiment is for allowing a subject to experience the sensation of a phantom limb for a limb on either the left or right side of the body while positioned in front of the apparatus.

In this second embodiment, the second chamber has the same constituent features as the first chamber, and has a structure symmetrical to the first chamber about a mirror. As a result, in this second embodiment, a configuration is employed capable of accommodating limbs on both the left and right sides of the body. Furthermore, the same reference symbols are used to indicate those constituent features of the second embodiment that are in common with those of the first embodiment, and duplicate explanations thereof are omitted. In addition, dashes (′) are added to those reference symbols for those constituent features of the second chamber that are the same as those of the first chamber, and duplicate explanations thereof are also omitted.

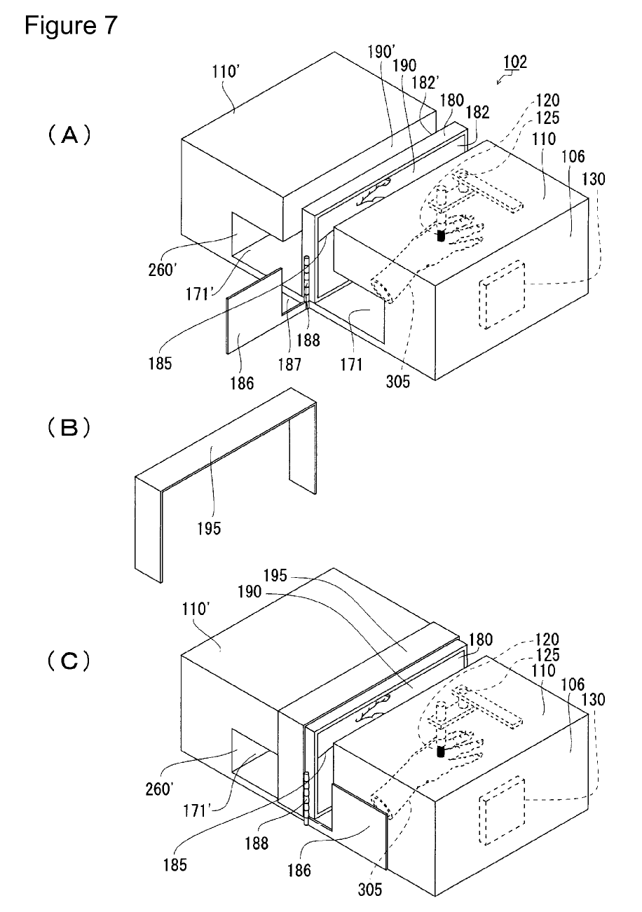

The following provides an explanation of the configuration of an apparatus in a second embodiment of the present invention with reference to FIGS. 7A to 7C, FIG. 8 and FIG. 9. Furthermore, FIGS. 7A to 7C, 8 and 9 are each drawings indicating an example of the configuration of an apparatus in the second embodiment.

FIG. 7A shows an external view of a case of the apparatus, FIG. 7B shows an external view of a cover attached to the case, and FIG. 7C shows an external view of the apparatus when the cover is attached to the case.

As shown in FIG. 7A, this apparatus 102 for experiencing visual-tactile interaction of the present embodiment (to be simply referred as the “apparatus”) has a first chamber 110 and a second chamber 110′ separated by a wall inside a case 106. The first chamber 110 and the second chamber 110′ are arranged in a row.

In this second embodiment, the first chamber 110 is a chamber for placing therein a prosthesis 305 that mimics a limb on the left side of the body as well as for inserting a limb on the right side of the subject's body. The first chamber 110 is provided with a stimulating unit 120 for giving a dynamic stimulus to the prosthesis 305, and a drive unit 125 (to be referred to as a “first drive unit”) that supports the stimulating unit 120 and allows the stimulating unit 120 to moving operation and/or turning operation (including a vibrating operation). In addition, the second chamber 110′ is a chamber for placing therein a prosthesis 305 that mimics a limb on the right side of the body as well as for inserting a limb on the left side of the subject's body. The second chamber 110′ is provided with a stimulating unit 120′ for giving a dynamic stimulus to the prosthesis 305, and a drive unit 125′ (to be referred to as a “second drive unit”) that supports the stimulating unit 120′ and allows the stimulating unit 120′ to cause moving operation and/or turning operation (including a vibrating operation). Thus, this apparatus 102 enables a person missing a limb on the left or right side of the body to use the apparatus while positioned on the front side of the apparatus, a person missing a limb on the left side of the body to use the apparatus while positioned on the front side of the apparatus, or a person missing a limb on the right side of the body to use the apparatus while positioned on the back side of the apparatus. In addition, this apparatus 102 can be used to have a subject having experienced the sensation of a phantom limb on the side of a missing limb while positioned on the front side of the apparatus to experience directly the stimulus of the stimulating unit 120 with an actual limb that is not missing while positioned on the back side of the apparatus, thereby allowing the subject to confirm the effects of experiencing the sensation of a phantom limb (this confirmation is carried out under the control conditions to be described later). This point will be described later.

Furthermore, with the exception of the portion of the viewing window 190 of the first chamber 101, the wall on the front side of the case 105 and the wall on the back side of the case 105 are closed in the first embodiment. In this second embodiment, however, as is clear from FIGS. 7A and 7B, a portion of the wall on the front side of case 106 and a portion of the wall on the back side of the case 106 of the first chamber 110 are opened by an opening 171. This opening 171 allows a right limb of a subject to be inserted into the first chamber 110, namely between the mirror 180 and the blocking plate 260. In addition, a portion of the wall on the front side of the case 106 and a portion of the wall on the back side of the case 106 of the second chamber 110′ are similarly opened by an opening 171′. This opening 171′ allows a left limb of a subject to be inserted into the second chamber 110′, namely between the mirror 180 and the blocking plate 260′. Furthermore, in the example shown in FIGS. 7A and 7C, these openings 171 and 171′ are formed into the shape of a “U” so as to be continuous with the viewing window 190 or 190′. However, these openings 171 and 171′ may also be formed into, for example, a rectangular shape so as to be separated from the viewing window 190 or 190′.

The mirror 180 is provided between the first chamber 110 and the second chamber 110′. In addition, the viewing window 190 (to be referred to as a “first viewing window”) is provided between the first chamber 110 and the mirror 180, while the viewing window 190′ (to be referred to as a “second viewing window”) is provided between the mirror 180 and the second chamber 110′.

In this second embodiment, a double-sided mirror, or in other words, a mirror having a mirrored surface on both sides thereof, is used for the mirror 180. The mirrored surface on the side opposing the first chamber 110 is to be referred to as “first mirrored surface 182”, while the mirrored surface on the side opposing the second chamber 110′ is to be referred to as “second mirrored surface 182”. Furthermore, the first mirrored surface 182 is a surface for reflecting the mirror image 185 of the prosthesis 305 placed inside the first chamber 110, while the second mirrored surface 182′ is a surface for reflecting a mirror image 185′ of the prosthesis placed inside the second chamber 110′.

In addition, in this second embodiment, the mirror 180 has at least a predetermined thickness, and is provided with a door 186 on the side of the front side. This door 186 is fastened to the mirror 180 by a hinge 188 so as to rotate to the left or right. This door 186 functions as a cover that blocks either opening 171 of the first chamber 110 or opening 171′ of the second chamber 110′. This door 186 blocks the opening 171 of the first chamber 110 by being rotated to the right side as shown in FIG. 7C in the case of allowing a subject to experience the sensation of a limb on the left side of the body.

This apparatus 102 allows the opening 171 or opening 171′, which is not used to experience the sensation of a phantom limb, to be selectively blocked by the door 186. Consequently, this apparatus 102 allows a subject to insert a limb to be used to experience the sensation of a phantom limb into the correct opening 171 or opening 171′ at all times.

Furthermore, this door 186 blocks the opening 171′ of the second chamber 110′ by rotating to the left side in the case of allowing a subject to experience the sensation of a phantom limb on the right side of the body. This door 186 is provided with a slit 187 so as to expose the viewing window 190 or 190′ in the state of blocking either the opening 171 of the first chamber 110 or the opening 171′ of the second chamber 110′. Furthermore, this door 186 may also be provided on the side of the rear side of the mirror 180.

The viewing window 190 or 190′ is blocked by a cover 195 (to be referred to as a “viewing window cover”) as shown in FIG. 7B. This viewing window cover 195 blocks the second viewing window 190′ on the left side as shown in FIG. 7C in the case of allowing a subject to experience the sensation of a phantom limb for a limb on the left side of the body. In addition, this viewing window cover 195 blocks the first viewing window 190 on the right side in the case of allowing a subject to experience the sensation of a phantom limb for a limb on the right side of the body.

Since a subject ends up being aware of the mirror image 185 in the case of having viewed the mirror image 185 that is not to be used to experience the sensation of a phantom limb, the subject is unable to adequate experience the sensation of a phantom limb. Thus, the mirror image 185 that is not to be used to experience the sensation of a phantom limb becomes an impairment to allowing a subject to experience the sensation of a phantom limb. Consequently, this apparatus 102 is able to conceal the mechanism not used to experience the sensation of a phantom limb from a subject by selectively blocking the viewing window 190 or 190′ that is not to be used to experience the sensation of a phantom limb. As a result, this apparatus 102 allows a subject to preferably experience the sensation of a phantom limb at all times.

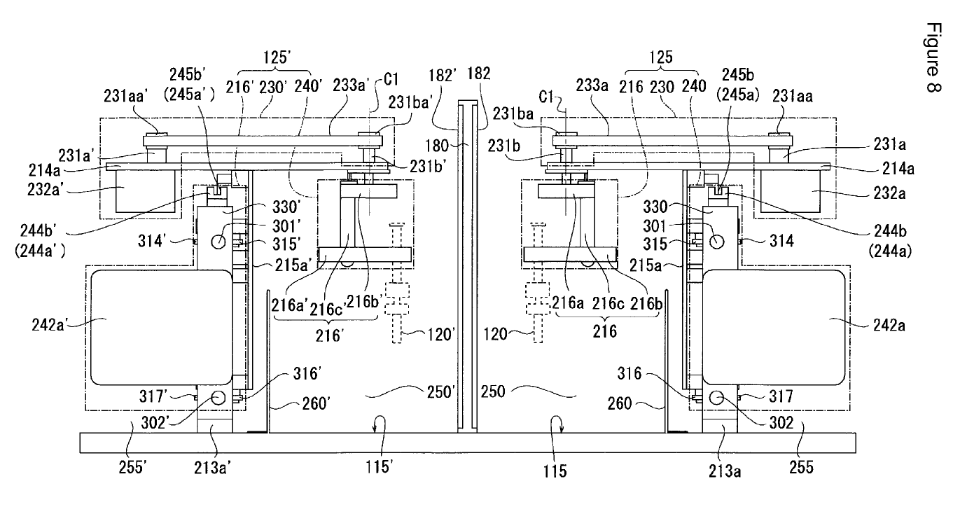

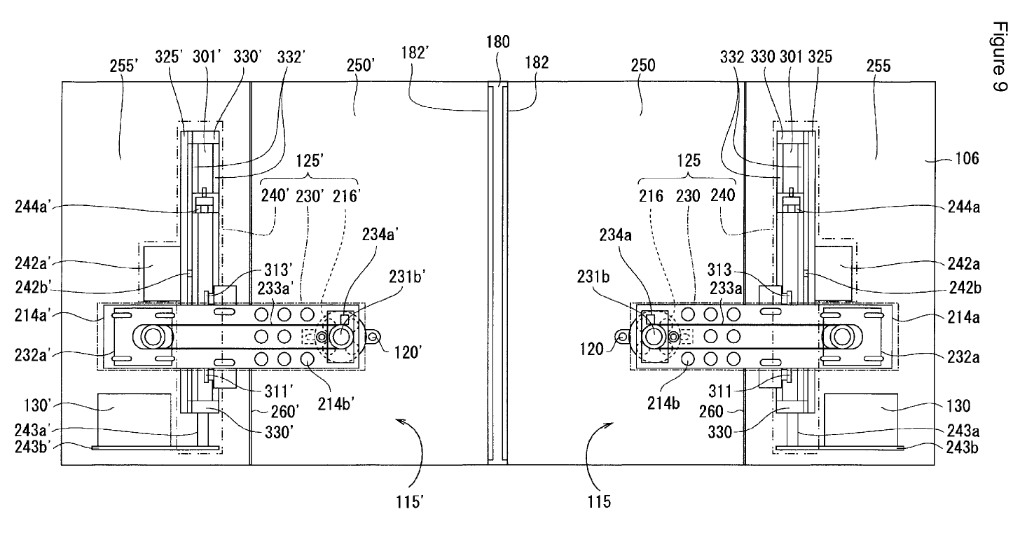

FIGS. 8 and 9 show examples of the configurations of the stimulating units 120 and 125′ and the drive units 125 and 125′ FIG. 8 shows the configurations of stimulating units 120 and 120′ and drive units 125 and 125′ as viewed from the front, while FIG. 9 shows the configurations of stimulating units 120 and 120′ and drive units 125 and 125′ as viewed from above.

Furthermore, the door 186 and the hinge 188 as shown in FIGS. 7A and 7C have been omitted from FIGS. 8 and 9.

The configuration of the stimulating unit 120 is as was previously explained in the first embodiment. This apparatus 102 secures a large space to allow a subject to insert a limb on the right side of the body by removing the stimulating unit 120 from the first drive unit 125. As a result, this apparatus 102 allows a subject to easily insert a limb on the right side of the body into the first chamber 110.

In addition, the configuration of the first drive unit 125 is as was explained in the first embodiment.

On the other hand, the configuration of the stimulating unit 120′ is the same as the configuration of the stimulating unit 120. This apparatus 102 secures a large space to allow a subject to insert a limb on the left side of the body by removing the stimulating unit 120′ from the second drive unit 125′. As a result, this apparatus 102 allows a subject to easily insert a limb on the left side of the body into the second chamber 110′.

In addition, the configuration of the second drive unit 125′ is the same as the configuration of the first drive unit 125.

In the present embodiment, the control unit 130 shown in FIG. 7C employs a configuration for controlling both the first drive unit 125 and the second drive unit 125′. The operation of the control unit 130 is switched by an operator of the apparatus 102 so as to control either the first drive unit 125 or the second drive unit 125′.

In this apparatus 102, a large space for allowing insertion of either limb is secured by removing the stimulating unit 120 of the first chamber 110 from the first drive unit 125, or by removing the stimulating unit 120′ of the second chamber 110′ from the second drive unit 125′.

In the case of experiencing the sensation of a phantom limb for a limb on the left side of the body, a subject inserts a limb of the subject on the left side of the body into the second chamber 110′ through the opening 171′ and places it on a flat surface 115′ (to be referred to as a “second flat surface”) of the second chamber 110′. Furthermore, in this case, the door 186 shown in FIGS. 7A and 7C is positioned on the right side of the mirror 180 so as to block the opening 171 of the first chamber 110. In addition, in this case, the viewing window cover 195 shown in FIGS. 7B and 7C is attached over the second viewing window 190′ so as to block the second viewing window 190′. When a subject places a limb on the left side of the body on the second flat surface 115′, the mirror image 185 of the prosthesis 305 given a dynamic stimulus by the stimulating unit 120 is viewed through the first viewing window 190 provided between the first chamber 110 and the mirror 180. As a result, this apparatus 102 allows the subject to experience the sensation of a phantom limb for a limb on the left side of the body.

On the other hand, in the case of experiencing the sensation of a phantom limb for a limb on the right side of the body, a subject inserts a limb of the subject on the right side of the body into the first chamber 110 through the opening 171, and places it on a flat surface 115 (to be referred to as a “first flat surface”) of the first chamber 110. Furthermore, in this case, the door 186 shown in FIGS. 7A and 7C is positioned on the left side of the mirror 180 so as to block the opening 171′ of the second chamber 110′. In addition, in this case, the viewing window cover 195 shown in FIGS. 7B and 7C is attached over the first viewing window 190 so as to block the first viewing window 190. When a subject places a limb on the right side of the body on the first flat surface 115, the mirror image 185′ of the prosthesis 305 given a dynamic stimulus by the stimulating unit 120′ is viewed through the second viewing window 190′ provided between the second chamber 110′ and the mirror 180. As a result, this apparatus 102 allows a subject to experience the sensation of a phantom limb for a limb on the right side of the body.

Thus, this apparatus 102 allows a subject positioned on the front side of the apparatus to experience the sensation of a phantom limb for limb on either the left or right side of the body.

Furthermore, the mirror 180 in this second embodiment is in the form of a double-sided mirror. However, a single-sided mirror can also be used for the mirror 180 by employing a structure that allows the mirror 180 to be removed from the case 106 and attached to the case 106 after changing the orientation thereof corresponding to the orientation of the chamber on the side in which the prosthesis 305 is placed so as to be opposed to the chamber on the side in which the prosthesis 305 is placed.

As has been described above, the apparatus 102 for experiencing visual-tactile interaction of the second embodiment allows a subject to experience the sensation of a phantom limb for a limb on either side of the body by having the subject alternately sit in opposition to the front side and back side of the apparatus, or by having the subject sit in opposition to the front side of the apparatus for each limb.

The apparatus 102 for experiencing visual-tactile interaction of the present embodiment is suitable for verifying control conditions. Furthermore, “control conditions” refer to conditions for actually demonstrating the desired effects, namely that the sensation of a phantom limb has actually been experienced by a subject in the case of this apparatus 102.

Verification of control conditions is carried out in the manner described below.

For example, a normal person, namely a person having all of his or her limbs intact without being missing, inserts any part of an actual limb of the normal person into the first chamber 110 or the second chamber 110′ of this apparatus 102. Here, the explanation uses the example of the normal person inserting his or her left arm into the second chamber 110′.

The apparatus 102 gives a dynamic stimulus from the stimulating unit 120′ to the left arm of the normal person by inserting the left hand into the second chamber 110′ arranging a prosthesis of the right hand in the first chamber 110. As a result, this apparatus 102 allows the normal person to experience the sensation of actually being given a dynamic stimulus.

Subsequently, the normal person removes the left hand from the second chamber 110′, and then views the mirror image 185 reflected in the first mirrored surface 182 of the mirror 180 through the first viewing window 190 while imagining the left wrist inserted into the second chamber 110′. Furthermore, the prosthesis 305 mimicking the right hand is placed in the first chamber 110 at this time, and the mirror image 185 of the right hand corresponding to the left hand is reflected in the first mirrored surface 182 of the mirror 180.

Next, the stimulating unit 120 of the apparatus 102 is operated and the stimulating unit 120′ of the apparatus 102 is not operated. When the normal person views the mirror image 185 through the first viewing window 190, a dynamic stimulus is given to the prosthesis 305 by the stimulating unit 120.

At this time, the apparatus 102 allows the subject in the form of a normal person to experience the sensation of a phantom limb for the left hand, namely the sensation of a dynamic stimulus being given by the stimulating unit 120′, in the case the drive conditions of the stimulating unit 120 coincide with the drive conditions of the stimulating unit 120′ when the subject was allowed to actually experience a dynamic stimulus.

On the other hand, this apparatus 102 is unable to allow a normal person to experience the sensation of a phantom limb in the case the drive conditions of the stimulating unit 120 do not coincide with the drive conditions of the stimulating unit 120′ when the subject was allowed to actually experience a dynamic stimulus. In this case, the drive conditions of the stimulating unit 120 of the apparatus 102 are changed. When the drive conditions of the apparatus 102 are changed, the normal person again views the mirror image 185 reflected in the first mirrored surface 182 of the mirror 180 through the first viewing window 190 while imagining his or her left arm inserted into the second chamber 110′. This apparatus 102 gives a dynamic stimulus from the stimulating unit 120 to the prosthesis 305 when the normal person views the mirror image 185 through the first viewing window 190. The apparatus 102 repeats this procedure until the normal person is able to experience the sensation of a phantom limb for the left hand.

In the case the normal person has been able to experience the sensation of a phantom limb for the left hand, the apparatus 102 repeats this procedure for the parts of other limbs, namely the right arm, left leg and right foot, as necessary. As a result, this apparatus 102 enables a subject in the form of a normal person to sequentially experience the sensation of a phantom limb for all limbs.

In this manner, verification of control conditions is completed once this apparatus 102 has been able to allow a normal person to experience the sensation of a phantom limb for a limb. Furthermore, verification of control conditions is only carried out for the left and right arms or hands in the case the apparatus 102 is only to be used for arms. In addition, verification of control conditions is only carried out for the left and right legs or feet in the case the apparatus 102 is only to be used for legs.

In this manner, once verification of control conditions has been completed, this apparatus 102 allows any person, such as a person missing a portion of a limb, for example, to effectively experience the sensation of a phantom limb.

Furthermore, the drive conditions of the stimulating unit obtained through verification of control conditions as described above can be applied to another apparatus such as the apparatus 102 of the present embodiment or the apparatus 100 of the previously described first embodiment.

The present invention is not limited to the above-mentioned embodiments, and various modifications or variations can be made within a range that does not deviate from the aspect of the present invention.

For example, in the examples indicated in the first and second embodiments, a support unit supports a stimulating unit perpendicular to a first flat surface, namely the bottom of a case. However, this support unit may be made to support the stimulating unit on an angle to the first flat surface.

In addition, although a stimulating unit is attached to a first plate-like member of a drive unit by means of a support in the previously described first and second embodiments, the stimulating unit may also be attached to the first plate-like member directly without the use of a support.

In addition, the drive unit may be configured so as to freely drive a stimulating unit by combining linear movement and turn within an XY coordinate system, or may be configured so as to freely drive this stimulating unit by combining linear movement and turn within an XY coordinate system.

This driving can be controlled with a computer, and in that case, various stimulation patterns can be stored in memory according to the various stimulation patterns given to the prosthesis, thereby enabling a stimulating unit to be operated in a selected drive pattern by operating the corresponding required mode operation switch, and the employing of a configuration that allows the control unit and drive unit to drive in this manner is both easy and simple for a person with ordinary skill in the art.

### INDUSTRIAL APPLICABILITY

The apparatus for experiencing visual-tactile interaction of the present invention can not only be used as a therapeutic apparatus for phantom pain, but can also be used as an educational apparatus for allowing subjects to experience the sensation of a phantom limb.

1. An apparatus for experiencing visual-tactile interaction, comprising:
a case having therein a first chamber for placing therein a prosthesis mimicking a part of either one of a left limb and a right limb, and a second chamber arranged in parallel with said first chamber and provided with an opening for allowing a subject to insert a part of the other one of the limbs corresponding to that of said prosthesis into said second chamber;
a stimulating unit, provided within said first chamber, for giving a dynamic stimulus to said prosthesis,
a drive unit, provided within said first chamber, for supporting said stimulating unit and allowing said stimulating unit to move and/or turn;
a mirror, provided perpendicular to the bottom of said case between said first chamber and said second chamber, for reflecting a mirror image of said prosthesis placed inside said first chamber, and
a viewing window provided in said case for allowing the subject to view a mirror image of the prosthesis reflected in the mirror, wherein
by allowing a subject to view a mirror image of said prosthesis which is being subjected to a dynamic stimulus by said stimulating unit through said viewing window, said subject is caused to experience sensation of a phantom limb produced by interaction between visual sensation and tactile sensation.

2. The apparatus for experiencing visual-tactile interaction according to claim 1, wherein said stimulating unit is formed as a contacting member that stimulates said prosthesis by direct contact.

3. The apparatus for experiencing visual-tactile interaction according to claim 1, wherein said stimulating unit is formed as a fluid sprayer that stimulates said prosthesis by spraying a fluid.

4. The apparatus for experiencing visual-tactile interaction according to claim 3, wherein in a case where said fluid sprayer sprays said fluid in a form of wind, said subject is allowed to view a mirror image of said prosthesis as if having been deformed in a manner of the human body by said wind, using said prosthesis formed from a resin having flexibility similar to that of the skin of a limb of the human body.

5. The apparatus for experiencing visual-tactile interaction according to claim 3, wherein in a case where said fluid sprayer sprays said fluid in a form of a liquid, said subject is allowed to view a mirror image of said prosthesis on which said liquid has been sprayed.

6. The apparatus for experiencing visual-tactile interaction according to claim 1, wherein said stimulating unit is formed as a light projecting unit that stimulates said prosthesis by projecting light.

7. The apparatus for experiencing visual-tactile interaction according to claim 1, wherein said first chamber comprises a blocking plate that conceals a portion of said drive unit so that the portion of said drive unit is not reflected in the area of said mirror viewed from said viewing window.

8. The apparatus for experiencing visual-tactile interaction according to claim 1, wherein
said drive unit comprises:
a support for supporting said stimulating unit;
a rotation mechanism for rotating said support;
a first plate-like member on which said rotation mechanism is installed for rotatably supporting said support;
a second plate-like member coupled to one end of said first plate-like member; and
a slide mechanism, attached to said second plate-like member, for moving said first plate-like member within a plane in parallel with the bottom of said case by means of said second plate-like member.

9. The apparatus for experiencing visual-tactile interaction according to claim 8, wherein said first plate-like member comprises a hole or groove for weight reduction.

10. The apparatus for experiencing visual-tactile interaction according to claim 1, wherein said opening of said second chamber is provided at a location that allows the part of said other one of the limbs of said subject corresponding to that of said prosthesis to appear to be overlapping said mirror image of said prosthesis placed in said first chamber when said other one of the limbs is inserted into said second chamber through said opening.

11. The apparatus for experiencing visual-tactile interaction according to claim 1, wherein said opening of said second chamber is provided on a front side and on a back side of said second chamber, respectively.

12. The apparatus for experiencing visual-tactile interaction according to claim 1, wherein said prosthesis is formed from a resin.

13. The apparatus for experiencing visual-tactile interaction according to claim 12, wherein said resin is a resin, such as synthetic rubber or natural rubber, having flexibility roughly the same as that of the skin of a limb of the human body.

14. An apparatus for experiencing visual-tactile interaction, comprising:
a case having therein a first chamber and a second chamber arranged in parallel;
a first stimulating unit, provided within said first chamber, for giving a dynamic stimulus to a first prosthesis, placed in said first chamber, that mimics a part of one of a left limb and a right limb;
a first drive unit, provided within said first chamber, for supporting said first stimulating unit and allowing said first stimulating unit to move and/or turn;
a second stimulating unit, provided within said second chamber, for giving a dynamic stimulus to a second prosthesis, placed in said second chamber, that mimics a part of the other left or right limb;
a second drive unit, provided within said second chamber, for supporting said second stimulating unit and allowing said second stimulating unit to move and/or turn,
a first opening, provided in said case, for allowing a subject to insert a part of one of the limbs corresponding to said second prosthesis into said first chamber;
a second opening, provided in said case, for allowing a subject to insert a part of the other one of the limbs corresponding to that said first prosthesis into said second chamber;
a mirror, provided perpendicular to the bottom of said case between said first chamber and said second chamber, for reflecting both a mirror image of said first prosthesis placed within said first chamber and a mirror image of said second prosthesis placed within said second chamber; and
a first viewing window of said first chamber for allowing said subject to view a mirror image of said first prosthesis reflected in said mirror, and a second viewing window of said second chamber for allowing said subject to view a mirror image of said second prosthesis reflected in said mirror, each of which is provided in said case, wherein
(a) when the prosthesis placed in the apparatus is said first prosthesis by allowing a subject, who places said first prosthesis in said first chamber and inserts said part of said the other one of the limbs corresponding to that of said first prosthesis into said second chamber, to view a mirror image of said first prosthesis which is being subjected to a dynamic stimulus by said first stimulating unit through said first viewing window, said subject is caused to experience sensation of a phantom limb produced by visual-tactile interaction, or
(b) when the prosthesis placed in the apparatus is said second prosthesis, by allowing a subject, who places and said second prosthesis in said second chamber and inserts said pat of said other one of the limbs corresponding to that of said second prosthesis into said first chamber, to view a mirror image of said second prosthesis which is being subjected to a dynamic stimulus by said second stimulating unit through said second viewing window, said subject is caused to experience sensation of a phantom limb produced by visual-tactile interaction.

15. An apparatus for experiencing visual-tactile interaction, comprising:
a case having therein a first chamber and a second chamber arranged in parallel;
a first stimulating unit, provided within said first chamber, for giving a dynamic stimulus to a first prosthesis, placed in said first chamber, that mimics a part of either one of a left limb and a right limb;
a first drive unit, provided within said first chamber, for supporting said first stimulating unit and allowing said first stimulating unit to move and/or turn;
a second stimulating unit, provided within said second chamber, for giving a dynamic stimulus to a second prosthesis, placed in said second chamber, that mimics a part of the other one of the left limb and the right limb;
a second drive unit, provided within said second chamber, for supporting said second stimulating unit and allowing said second stimulating unit to move and/or rotate;
a first opening, provided in said case, for allowing a subject to insert a part of one of the limbs corresponding to that of said second prosthesis into said first chamber;
a second opening, provided in said case, for allowing a subject to insert a part of the other one of the limbs corresponding to that of said first prosthesis into said second chamber;
a mirror, provided perpendicular to the bottom of said case between said first chamber and said second chamber, for reflecting either a mirror image of said first prosthesis placed within said first chamber or a mirror image of said second prosthesis placed within said second chamber; and
a first viewing window of said first chamber for allowing said subject to view a mirror image of said first prosthesis reflected in said mirror, and a second viewing window of said second chamber for allowing said subject to view a mirror image of said second prosthesis reflected in said mirror, each of which is provided in said case, wherein
said mirror has a structure enabling said mirror to be removed from said case and attached to said case after changing the orientation thereof, and
(a) when the prosthesis placed in the apparatus is said first prosthesis, by allowing a subject, who places said first prosthesis in said first chamber, with said mirror being attached to said case so as to oppose said first chamber, and who inserts said part of said other one of the limbs corresponding to that of said first prosthesis into said second chamber, to view a mirror image of said first prosthesis which is being subjected to a dynamic stimulus by said first stimulating unit through said first viewing window, and said subject is caused to experience sensation of a phantom limb produced by visual-tactile interaction, or
(b) when the prosthesis placed in the apparatus is said second prosthesis, by allowing a subject, who places said second prosthesis in said second chamber, with said mirror being attached to said case so as to oppose said second chamber, and who inserts said part of said other one of the limbs corresponding to that of said second prosthesis into said first chamber, to view a mirror image of said second prosthesis which is being subjected to a dynamic stimulus by said second stimulating unit through said second viewing window, and said subject is caused to experience sensation of a phantom limb produced by visual-tactile interaction.

16. The apparatus for experiencing visual-tactile interaction according to claim 14, further comprising a cover for selectively closing said first viewing window or said second viewing window.

17. The apparatus for experiencing visual-tactile interaction according to claim 14, further comprising a cover for selectively closing the opening of said first chamber or the opening of said second chamber.

18. The apparatus for experiencing visual-tactile interaction according to claim 15, further comprising a cover for selectively closing said first viewing window or said second viewing window.

19. The apparatus for experiencing visual-tactile interaction according to claim 15, further comprising a cover for selectively closing the opening of said first chamber or the opening of said second chamber.
